(19) United States
(12) Patent Application Publication
(10) Pub. No.: US 2009/0042937 A1
(43) Pub. Date: Feb. 12, 2009

(54) NITROXIDES FOR USE IN TREATING OR PREVENTING AMYLOID-RELATED DISEASES

(76) Inventors: Louis Habash, Newport Beach, CA (US); Clarence Jones, Huntington Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/815,436

(22) PCT Filed: Feb. 2, 2006
(86) PCT No.: PCT/US2006/003974

§ 371 Date: Sep. 3, 2008

Related U.S. Application Data
(60) Provisional application No. 60/649,288, filed on Feb. 2, 2005.

(51) Int. Cl. A61K 31/445; A61P 25/28

(52) U.S. Cl. 514/315

(57) ABSTRACT

Phamaceutical compositions are provided that are useful in treating amyloid protein-related diseases such as Alzheimer's disease. The compositions comprise a pharmaceutically acceptable carrier, and an effective therapeutic or prophylactic amount of a nitroxide antioxidant that downregulates one or more genes related to the amyloid-related disease. Methods are also provided for the use of the pharmaceutical compositions in the treatment or prevention of amyloid protein-related diseases. In a preferred embodiment, the nitroxide antioxidant is Tempol (4-hydroxy-2,2,6,6-tetramethylpiperidine-1-oxyl), and the amyloid protein-related disease is Alzheimer's disease. In a preferred embodiment, the downregulated genes are genes related to inflammation or oxidative stress.

### BACKGROUND OF THE INVENTION

2. Field of the Invention

The present invention relates to pharmaceutical compositions useful for treating or preventing amyloid-related diseases such as Alzheimer's disease, and to methods for using these compositions in treating or preventing such conditions.

2. Description of the Related Art

Alzheimer's disease is the most common form of dementia in the aged. It is characterized by mental deterioration and cognitive decline, and is the third leading cause of death after heart disease and cancer. Additionally, the prevalence of the disease increases exponentially with age: the incidence doubles every 5 years between 65 and 85, and affects one of every five people by the age of 80. The disease has become a major health problem in the United States, with 4.5 million people affected.

Alzheimer's disease is a chronic inflammatory disorder characterized by the appearance in the brain of senile plaques and neurofibrillary tangles, with the former being somewhat pathognomonic for Alzheimer's disease and the latter seen in other neurodegenerative diseases. Senile plaques are also termed amyloid plaques or deposits, as they are comprised primarily of β amyloid peptide (Aβ) plus other fibrillar elements including serum amyloid A and serum amyloid P. Aβ is a proteolytic byproduct of a much larger transmembrane protein known as the amyloid protein precursor (APP). Both senile plaques and neurofibrillary tangles are associated with the widespread neurodegeneration that is a characteristic of Alzheimer's disease. It is presently not known whether the amyloid deposits themselves are the cause of neuronal loss. The loss of neurons is, however, associated with inflammation and oxidative stress markers. A schematic of proposed possible amyloid-related pathways leading to the development of Alzheimer's disease is shown in FIG. 1. (Veurink et al., Ann. Hum. Biol. 30(6): 639-667)

Furthermore, the neurofibrillary tangles chiefly comprise phosphorylated Tau protein; hyperphosphorylation of the Tau protein results from aberrant regulation of kinases and phosphatases. Normally, the Tau protein functions to stabilize microtubules, but when hyperphosphorlyated, it cannot perform this function, and instead aggregates and forms tangles. These tangles lead to neuronal degeneration and cell death. The Tau protein is also subject to cleavage by caspases. There is also some evidence that apoptosis plays a role in neuronal loss.

Further inflammatory mechanisms thought to play a part in the etiology of Alzheimer's disease include the complement system, the pentraxins, cytoidnes such as various interleuldns and TNF-α, the prostaglandins, the metallomatrix proteinases, components of the coagulation pathways, and the cathepsins.

In addition to the inflammatory mechanisms described above, recent theories suggest that oxidative stress also plays a part in the development of Alzheimer's disease. Oxidative stress results from an imbalance between antioxidant levels and the level of reactive species, such as the superoxide anion radical, hydrogen peroxide, and the hydroxyl radical. In Alzheimer's disease, oxidative stress appears to precede the specific cellular and tissue damage in the disease progression. Several studies have shown that dying neurons in AD patients are subject to high levels of oxidative stress, and that senile plaques are a focus of cellular and molecular oxidation.

Additionally, elevated levels of serum amyloid A are a hallmark of several other chronic inflammatory conditions that are thought to predispose affected patients to amyloidosis, the extracellular deposition of amyloid fibrils. These diseases include rheumatoid arthritis, juvenile chronic arthritis, psioratic arthropathy, ankylosing spondylitis, Behcet's syndrome, reactive arthritis and Crohn's disease (Cunnane et al., Bailliere's Clin. Rheumatol. 13:4 (1999) 615-628; Malle et al., Eur. J. Clin. Invest. 26 (1996) 427-435).

Prior approaches to preventing or altering the clinical course of chronic inflammatory disease and oxidative stress-related disease have generally been restricted to targets (or their precursors) directly associated with the etiopathogenesis of the condition, rather than to altering the expression of the genes encoding for these targets.

### SUMMARY OF THE INVENTION

Pharmaceutical compositions are provided that are useful in treating amyloid-related conditions. The compositions comprise a pharmaceutically acceptable carrier, and an effective therapeutic or prophylactic amount of an agent that down regulates one or more genes related to the amyloid-related disease. Methods are also provided for the use of the pharmaceutical compositions in the reduction of serum levels of amyloid proteins and in the treatment or prevention of amyloid-related diseases. In a preferred embodiment, the agent is a nitroxide antioxidant, such as Tempol (4-hydroxy-2,2,6,6-tetramethylpiperidine-1-oxyl), and the amyloid-related disease is Alzheimer's disease. In a preferred embodiment, the downregulated genes are genes related to inflammation, oxidative stress, apoptosis, or the abnormal proteins found in Alzheimer's patients.

### BRIEF DESCRIPTION OF THE DRAWINGS

FIG. 1 is a schematic diagram of proposed pathways in the development of Alzheimer's disease.

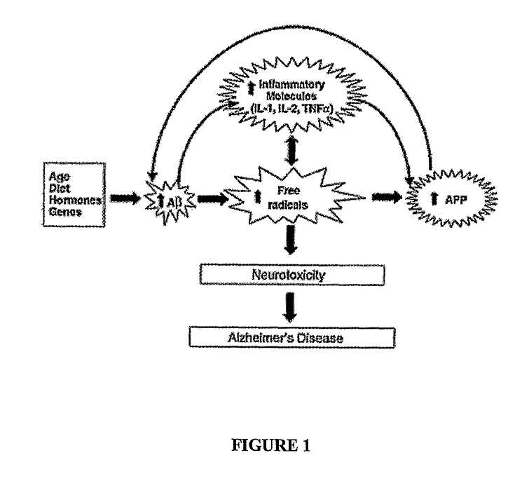

FIG. 2 is a schematic diagram showing the role of certain eukaryotic translation initiation factors in the intracellular level of Tau protein.

FIG. 3 is a schematic diagram showing the role of the cyclooxygenases in the generation of Aβ peptides.

### DETAILED DESCRIPTION OF THE PREFERRED EMBODIMENT

As described above, a composition and method are disclosed which are useful in treating or preventing an amyloid-related neurodegenerative disorder such as Alzheimer's disease. In a preferred embodiment, the agent used to down regulate genes related to neurodegenerative disorders is a nitroxide antioxidant. Tempol is a stable nitroxide radical characterized by the chemical formula 4-hydroxy-2,2,6,6-tetramethylpiperidine-1-oxyl that has antioxidative properties. The present applicants have discovered that Tempol possesses the novel property of down-regulating the genes encoding for co-participants in the pathogenesis of amyloid-related diseases such as Alzheimer's disease (see Tables 1 and 2 below). As described above, previous therapies have generally not focused on altering the expression patterns of disease-related genes.

Tempol accordingly exhibits a novel and unique therapeutic bimodality in such diseases: not only does it target the down-stream inflammatory products (directly reducing oxidative stress), but by down regulating the expression of associated genes, it also affects the upstream source of several other implicated species, such as serum amyloid A and serum amyloid P.

The use of other nitroxide compounds is also contemplated. According to certain embodiments the nitroxide compound can be selected from the following formulas:

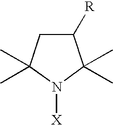

Wherein X is selected from O and OH, and R is selected from COOH, CONH, CN, and CH₂NH₂.

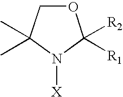

Wherein X is selected from O and OH, and R₁ is selected from CH₃ and spirocyclohexyl, and R₂ is selected from C₂H₅ and spirocyclohexyl.

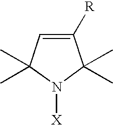

Wherein X is selected from O and OH, and R is selected from CONH.

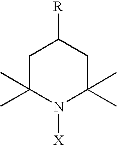

Wherein X is selected from O and OH and R is selected from H, OH, and NH₂.

Suitable nitroxide compounds can also be found in Proctor, U.S. Pat. No. 5,352,442, and Mitchell et al., U.S. Pat. No. 5,462,946, both of which are hereby incorporated by reference in their entireties.

A non-limiting list of nitroxide compounds include: 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, and 4-Oxo-TEMPO. TEMPO can also be substituted, typically in the 4 position, for example, 4-amino, 4-(2-bromoacetamido), 4-(ethoxyfluorophosphonyloxy), 4-hydroxy, 4-(2-iodoacetamido), 4-isothiocyanato, 4-maleimido, 4-(4-nitrobenzoyloxyl), 4-phosphonooxy, and the like.

### Downregulated Genes

To assess the effects of Tempol on gene expression, Tempol was administered to experimental mice at a dose of 5 mg/g of food from 14 months to 31 months after birth. Mice receiving the same food without the addition of Tempol were used as a negative control. At the age of 31 months, the experimental animals were sacrificed and the hearts were surgically removed. The expression of a broad spectrum of genes in the cardiac tissue was assessed using chip-based microarray technology. Such chips are well known in the art and are widely used to assess gene expression. The experimental results showed that certain genes related to inflammatory pathways implicated in amyloid-related disorders such as Alzheimer's disease exhibited a twofold or greater reduction in expression. These genes are shown in Table 1.

TABLE 1
INFLAMMATION-RELATED GENES DOWNREGULATED IN
CARDIAC TISSUE BY TEMPOL ADMINISTRATION

| | | Control mice | | | TEMPOL-treated mice | | | |
|---|---|---|---|---|---|---|---|---|
| ORF | Description | tpc1 | tpc2 | tpc3 | tp51 | tp52 | tp53 | Fold change |
| M63690 | Interferon γ-inducible | 32 | 25 | 7 | 29 | 20 | 1 | −2.0 |
| | protein/IRG 47GTP- | | | | | | | |
| | binding | | | | | | | |
| U15636 | T-cell specific | 211 | 174 | 299 | 146 | 79 | 164 | −2.0 |
| | GTPase | | | | | | | |
| AA116993 | Cyclophilin A | 9 | 22 | 13 | 12 | −14 | −10 | −2.1 |
| AA120298 | Immunophilin | 251 | 138 | 180 | 200 | 154 | 164 | −2.1 |
| | (FKBP25) | | | | | | | |
| X97227 | CD53 antigen | 35 | 36 | 27 | 21 | −17 | −13 | −2.1 |
| AA120109 | Interferon α-induced | 329 | 331 | 141 | 149 | 88 | 72 | −2.3 |
| | 11.5 kd protein (P27) | | | | | | | |
| M13521 | Serum amyloid A-1 | 80 | 57 | 44 | 6 | 1 | 8 | −2.5 |
| | protein precursor | | | | | | | |
| J03776 | T-lymphocyte 1/rpt-1 | 55 | 75 | 85 | 17 | 42 | 29 | −2.7 |
| | regulatory protein | | | | | | | |
| AA002526 | Interferon-inducible | 66 | 86 | 126 | 26 | 34 | 36 | −2.7 |
| | protein | | | | | | | |
| Y00426 | Serum amyloid P- | 283 | 284 | 169 | 43 | 61 | 119 | −2.8 |
| | component precursor | | | | | | | |
| | (SAP) | | | | | | | |
| M55561 | CD52 antigen | 109 | 363 | 223 | 106 | 177 | 171 | −3.5 |
| AA013783 | Interferon induced | 42 | 81 | 86 | −81 | 27 | 41 | −4.9 |
| | protein | | | | | | | |
| AA066354 | JAK-1 | 73 | 116 | 55 | 124 | 22 | 30 | −5.1 |
| W13546 | Lymphocyte | 1251 | 1145 | 1180 | −16 | −66 | 1331 | −55.2 |
| | receptor/polyubiquitin | | | | | | | |

Additionally, the results showed that the administration of Tempol also resulted in significant down regulation of genes related to oxidative stress, as shown in Table 2.

TABLE 2
OXIDATIVE STRESS-RELATED GENES DOWNREGULATED
IN CARDIAC TISSUE BY TEMPOL ADMINISTRATION

| | | Control mice | | | TEMPOL-treated mice | | | |
|---|---|---|---|---|---|---|---|---|
| ORF | Description | tpc1 | tpc2 | tpc3 | tp51 | tp52 | tp53 | Fold change |
| M32032 | Selenium-binding | 1362 | 1567 | 1702 | 554 | 600 | 670 | −2.1 |
| | protein 2 | | | | | | | |
| W62326 | Hsp-90 homolog | 36 | 54 | 59 | 6 | 16 | 39 | −2.4 |
| M65270 | Cathepsin B | 10 | 8 | 12 | −12 | 6 | 13 | −2.8 |
| W62091 | XPE UV-damaged | 249 | 280 | 233 | 61 | 75 | 213 | −2.9 |
| | DNA binding factor | | | | | | | |
| AA104319 | XPE-DNA damage | 114 | 188 | 127 | 25 | 37 | 115 | −5.2 |
| | binding protein | | | | | | | |

In a further gene expression study, Tempol was administered to experimental mice at a dose of 5 g/kg of diet from 12 months through 15 months. Mice receiving the same diet without the addition of Tempol were used as a negative control. At the age of 15 months, the adipose tissue of the experimental animals was obtained. The expression of a broad spectrum of genes in the adipose tissue was assessed using chip-based microarray technology. Specifically, in this case an Affymetrix MOE430A 2.0 array, containing 12,960 genes, was employed. Such chips are well known in the art and are widely used to assess gene expression. The experimental results on the adipose tissue show that certain genes related either to the levels of proteins implicated in Alzheimer's disease, or to apoptosis, exhibited an altered expression pattern. These genes are shown in Tables 3 and 4.

TABLE 3
GENE UPREGULATED IN ADIPOSE TISSUE
BY TEMPOL ADMINISTRATION

| | Mean (Control | Mean (Tempol- | P | Fold |
|---|---|---|---|---|
| Description | mice) | treated mice) | Value | change |
| Eukaryotic translation | 3685 | 4737 | 0.049 | 1.29 |
| initiation factor for | | | | |
| E binding protein 1 | | | | |
| (EIF4EBP1) | | | | |

TABLE 4
GENES DOWNREGULATED IN ADIPOSE
TISSUE BY TEMPOL ADMINISTRATION

| | Mean (Control | Mean (Tempol- | P | Fold |
|---|---|---|---|---|
| Description | mice) | treated mice) | Value | change |
| Eukaryotic translation | 1085 | 873 | 0.001 | −1.23 |
| initiation factor 4 | | | | |
| E2 (EIF4E2) | | | | |
| Caspase 3 | 604 | 414 | 0 | −1.47 |
| Cyclooxygenase 1 | 392 | 331 | 0.006 | −1.18 |
| (COX-1) | | | | |

A short summary of selected genes described in Tables 14, and their relevance to Alzheimer's disease and other amyloid-related disease is provided below.
1. Serum Amyloid A-1 Protein Precursor

Serum amyloid A-1 protein precursor is the precursor protein to serum amyloid A (SAA). Serum aniyloid A is an acute phase reactant whose precise biological function is largely unknown, but likely involves the promotion of chemotaxis, cellular adhesion, cytolcine production, and metalloproteinase secretion, all components of the inflammatory response. High levels of serum amyloid A are seen in patients with acute/chronic inflammation, and with prolonged or repeated inflammatory conditions, in which SAA remains elevated over time and predisposes patients to secondary amyloidosis. Secondary amyloidosis results from the deposition of serum amyloid A fibrils in various tissues, and is a frequent concomitant of several chronic inflammatory disorders including rheumatoid arthritis (Cunnane et al., Bailliere's Best Clin. Pract. Res. Clin. Rheumatol. 13:615-28 (1999)), familial Mediterranean fever (Ozen, Eur. J. Pediatr. 162:449-54 (2003)), atherosclerotic diseases (Hatters et al., Eur. Biophys. J. 31:2-8 (2002)), Alzheimer's disease, Parkinson's disease, end-stage renal disease (Kaysen et al., J. Ren. Nutr. 13:67-73 (2003)), juvenile chronic arthritis, psioratic arthropathy, ankylosing spondylitis, Behcet's syndrome, reactive arthritis, and Crohn's disease (Malle et al., Eur. J. Clin. Invest. 26:427-435 (1996)). It has been suggested that secondary amyloidosis might be prevented through suppression of the acute phase response and normalization of serum amyloid A levels (Cunnane, Curr. Opin. Rheumatol. 13:67-73 (2001)).

As shown in Table 1, the expression of serum ainyloid A-1 protein precursor in the cardiac tissue of the experimental mice was reduced 2.5-fold in the animals treated with Tempol.
2. Serum Amyloid P component precursor

Serum ainyloid P component precursor is a precursor protein to serum amyloid P (SAP). SAP is a plasma glycoprotein that is synthesized in the liver and circulates in normal human blood at a concentration of 30 to 45 mg/L (Pepys et al., Clin. Exp. Immunol. 32:119-124 (1978)). Although its biological function has yet to be elucidated, it is known to bind to a variety of ligands in a calcium-dependent fashion, including the complement component Clq (Bristow et al., Mol. Immunol. 23:1045-52 (1986); Ying et al., J. Immunol. 150:169-176 (1993)), C-reactive protein (Swanson et al., Biochim. Biophys. Acta. 1160:309-316 (1992); Christner et al., J. Biol. Chem. 269:9760-9766 (1994)), fibronectin (de Beer et al., J. Exp. Med. 154:1134-1139 (1981)), and fibrils in all types of amyloid deposits (Pepys et al., Ann. N.Y. Acad. Sci. 389:286-298 (1982)). It has also been localized in human atherosclerotic lesions (Li et al., Atheroscler. Thromb. Vasc. Biol. 15:22-57 (1995)).

Serum amyloid P is believed to contribute to the pathogenesis of amyloid deposition disorders (Pepys et al., Nature 417:254-59 (2002); Hutchinson et al., Mol. Med. 6:482-93 (2000)), including Alzheimer's, perhaps through protection of amyloid plaques from degradation thereby allowing for protraction of the plaque-associated inflammatory response (Herbert et al, Mol. Med. 8:9-15 (2002); McGeer et al., Sci. Aging Knowledge Environ. 29:re3 (2002)). Currently, there is considerable interest in therapies which would either deplete SAP from the host circulation or inhibit the formation of amyloidogenic fibrils (Pepys et al., Nature (2002); Tanzi et al., Neuron 43:605-8 (2004)).

As shown in Table 1, the expression of serum amyloid P component precursor in the cardiac tissue of the experimental mice was reduced 2.8-fold in the animals treated with Tempol.
3. JAI(-1 (TNF-alpha signaling/IL-2 signaling)

Janus kinase 1 (JAK-1) is a tyrosine protein kinase that activates signal transducers and activators of transcription (STAT) following the binding of TNF-A and interleukins (IL-2, IL-6) to certain cells (Miscia et al., Cell Growth Differ. 13:13-18 (2002); Rawat et al., Blood 96:3514-21 (2000)). IL-6 and TNF-α are thought to be highly influential in promoting the transcription of acute phase serum amyloid A-1 protein precursor. (Malle et al., Eur. J. Clin Invest. 26:427-435 (1996)). JAI(-STAT signaling is also elicited by an Abl oncoprotein (v-Abl) (Danial et al., Oncogene 19:2523-31 (2000)). In a recent of study of adjuvant-induced arthritis in rats (model for rheumatoid arthritis), chemokines CCR1, 2, and 5 activated STAT-1 and 3 through JAK-1 mediated phosphorylation at a time (day 18) which coincided with the peak inflammatory response in the joints of the experimental animals (Shahrara et al., Arthritis Rheum. 48:3568-83 (2003)). The authors of this latter report have concluded that the down-stream signaling proteins associated with CC receptors may be useful targets to control inflammation in rheumatoid arthritis. Down regulation of JAK-1, which triggers STAT activation following receptor binding, will therefore be of clinical utility not only in reducing the stimulatory effect of IL-6 and TNF-α on the transcription of serum amyloid A-1 protein precursor but also in ameliorating the effects of inflammatory processes in the development of Alzheimer's disease.

As shown in Table 1, the expression of JAI(-1 in the cardiac tissue of the experimental mice was reduced 5.1-fold in the animals treated with Tempol.
4. Cathepsin B

Cathepsin B is a papain-family cysteine protease that is located in lysosomes where it is involved in protein turnover and maintenance of normal cellular metabolism. Increased expression of the gene encoding for cathepsin B, resulting in a corresponding increase in levels of this enzyme, is observed in amyloid-related diseases such as rheumatoid arthritis (Yan et al., Biol. Chem. 384:845-54 (2003)). Downregulation of cathepsin B may, therefore, be useful in other amyloid-related diseases such as Alzheimer's disease.

As shown in Table 2, the expression of cathepsin B in the cardiac tissue of the experimental mice was reduced 2.8-fold in the animals treated with Tempol.
5. Eukaryotic Translation Initiation Factor System (EIF4 EBP 1, EIF4E2)

The eukaryotic initiation factors (EIFs) participate in and control the process of protein synthesis, which requires three stages of MRNA translation, termed initiation, elongation, and termination. One of the steps in the initiation of translation involves the formation of an EIF4F complex, which recruits ribosomal subunits to the mRNA (cap-dependent translation). The cap-binding pocket of EIF4E interacts with a cap moiety positioned at the extreme 5′ end of the MRNA. Formation of the EIF4F complex is antagonized by EIF4E binding proteins (Tee et al., FEBS Letters 564 (2004) 58-62).

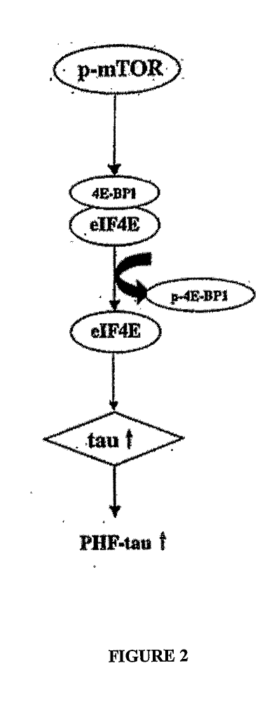

Furthermore, a checkpoint protein kinase known as mammalian target of rapamycin (mTOR) is an important effector of cell growth and proliferation, and exerts these effects via the regulation of protein synthesis. Specifically, and with respect to the eukaryotic translation initiation factors, the EIF4E binding protein is a direct target of mTOR, which phosphorylates and disassociates the EIF4E binding protein from EIF4E, which results in the activation of the EIF4E. It has recently been shown that the phosphorylated forms of EIF4E binding protein 1 and mTOR are dramatically increased in the brains of Alzheimer's patients, and have been localized inumohistochemically to neurons, along with phosphorylated Tau (Li et al., FEBS Journal 272 (2005) 4211-4220). A schematic view of this system is shown in FIG. 2. Because the high levels of phosphorylated mTOR in the brain of Alzheimer's patients lead to an increase in phosphorylated Tau protein, it would be desirable to alter the expression patterns of proteins in this system so as to reduce the amount of phosphorylated Tau available to form neurofibrillary tangles.

As shown in Tables 3 and 4, the expression of EIF4E binding protein 1 was upregulated 1.29-fold as a result of the administration of Tempol, while the expression of the EIF4E2 protein was downregulated 1.23-fold. This increase in the expression of the inhibitory binding protein, together with a decrease in the translation initiation factor itself, would be expected to reduce the amount of phosphorylated Tau protein in neurons.

### Cyclooxygenase-1 (COX-1)

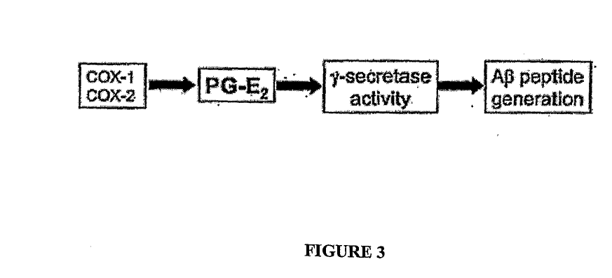

Cyclooxygenases are rate limiting enzymes necessary for the production of prostaglandins. There is evidence that COX-1 expression is elevated in the brain of Alzheimer's disease patients. It was suggested in a recent study that COX-1 may enhance Aβ peptide generation through a mechanism involved prostaglandin E2-mediated potentiation of gamma secretase activity (Qin et al., Journal of Biological Chemistry 278:51 (2003) 50970-50977). A schematic view of this system is shown in FIG. 3. Accordingly, downregulation of cyclooxygenase in Alzheimer's patients may be useful in reducing the amount of abnormal protein available to form senile plaques.

As shown in Table 4, the expression of COX-1 in the adipose tissue of the experimental mice was reduced 1.18-fold in the animals treated with Tempol.

### Caspase 3

The caspases are cystenyl endoproteases that cleave after aspartic acid residues. They are involved in apoptosis, and are expressed in the cytosol and exist as proenzymes. Upon apoptotic insult, the proenzyme caspases are cleaved to form active enzymes. A subset of the caspases are called effector caspases because once activated, they cleave several cellular proteins, thereby fatally altering cellular function. Caspase 3 is the main effector caspase and has received the most attention in the study of neuronal cell death. Caspase 3 appears to be involved in the cleavage of amyloid β4A precursor, which is associated with neuronal death in Alzheimer's disease, and an activated caspase 3 has been shown to be colocalized with caspase cleaved amyloid precursor protein in the granules of granulovacuolar degeneration in Alzheimer's disease (Su et al., Acta Neuropathol 104 (2002) 1-6). Furthermore, the Tau protein has also been shown to be cleaved by caspase 3 (Canu et al., Journal of Neuroscience 18 (1998) 7601-7074). The caspase 3-cleaved Tau has been shown to more rapidly assemble into Tau filaments that are the constituents of neurofibrillary tangles than wild-type Tau. Finally, as the major effector protein involved in apoptosis, caspase 3 is involved in neuronal cell death in response to the presence of amyloid β proteins (Awasthi et al., Experimental Neurology 196 (2005) 282-289). Downregulation of caspase 3 may, therefore, be useful in attenuating several of the steps leading to the development of Alzheimer's disease.

As shown in Table 4, the expression of caspase 3 in the adipose tissue of the experimental mice was reduced 1.47-fold in the animals treated with Tempol.

### Preferred Embodiment: Alzheimer's Disease Prophylaxis and Treatment Protocol

As described above, Tempol has the effect of altering the expression of genes related to inflammation, oxidative stress apoptosis, and pathological proteins that are implicated in Alzheimer's disease. Since the expression of these genes are altered, administration of Tempol will have a beneficial effect both by lessening the activity of the inflammatory and oxidative stress pathways which can lead to the development of Alzheimer's disease, as well as reducing the proteins involved in disease progression and neuronal cell death. In a preferred embodiment of the present invention, therefore, Tempol is administered to a mammalian host, such as a human, exhibiting no symptoms of dementia in order to prevent the development of Alzheimer's disease. Particularly preferred patients are those who are predisposed or otherwise at risk for Alzheimer's disease, such as those with a family history of the disease, or those with elevated APP levels or incipient plaques, or those with genetic or serum markers associated with the disease. Alternatively, Tempol may be administered to a human exhibiting symptoms of dementia or other evidence of disease initiation or progression, in order to retard or arrest the progress of Alzheimer's disease. For this purpose, Tempol, non-toxic salts thereof, acid addition salts thereof or hydrates thereof may be administered systemically or locally, usually by oral or parenteral administration.

The doses to be administered are determined depending upon, for example, age, body weight, symptom, the desired therapeutic effect, the route of administration, and the duration of the treatment. In the human adult, the dose per person at a time is generally from about 0.01 to about 1000 mg, by oral administration, from one to four or more times per day. Specific examples of particular amounts contemplated via oral administration include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 or more mg. The dose per person at a time is generally from about 0.01 to about 300 mg/kg via parenteral administration (preferably intravenous administration), from one to four or more times per day. Specific examples of particular amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more mg/kg. Continuous intravenous administration is also contemplated for from 1 to 24 hours per day to achieve a target concentration from about 0.01 mg/L to about 100 mg/L. Specific examples of particular amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mg/L. The dose to be used does, however, depend upon various conditions, and there may be cases wherein doses lower than or greater than the ranges specified above are used.

Tempol may be administered in the form of, for example, solid compositions, liquid compositions or other compositions for oral administration, injections, liniments or suppositories for parenteral administration.

Solid compositions for oral administration include compressed tablets, pills, capsules, dispersible powders and granules. Capsules include hard capsules and soft capsules. In such solid compositions, Tempol may be admixed with an excipient (e.g. lactose, mannitol, glucose, microcrystalline cellulose, starch), combining agents (hydroxypropyl cellulose, polyvinyl pyrrolidone or magnesium metasilicate aluminate), disintegrating agents (e.g. cellulose calcium glycolate), lubricating agents (e.g. magnesium stearate), stabilizing agents, agents to assist dissolution (e.g. glutamic acid or aspartic acid), or the like. The agents may, if desired, be coated with coating agents (e.g. sugar, gelatin, hydroxypropyl cellulose or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups and elixirs. In such compositions, Tempol is dissolved, suspended or emulsified in a commonly used diluent (e.g. purified water, ethanol or mixture thereof). Furthermore, such liquid compositions may also comprise wetting agents or suspending agents, emulsifying agents, sweetening agents, flavoring agents, perfuming agents, preserving agents, buffer agents, or the like.

Ejections for parenteral administration include solutions, suspensions, emulsions and solids which are dissolved or suspended. In injections, Tempol may be dissolved, suspended and emulsified in a solvent. The solvents are, for example, distilled water for injection, physiological salt solution, vegetable oil, propylene glycol, polyethylene glycol, alcohol such as ethanol, or a mixture thereof. Moreover the injections may also include stabilizing agents, agents to assist dissolution (e.g. glutamic acid, aspartic acid or POLYSORBATE80 (registered trade mark)), suspending agents, emulsifying agents, soothing agents, buffer agents, preserving agents, etc. They are sterilized in the final process or manufactured and prepared by sterile procedure. They may also be manufactured in the form of sterile solid compositions, such as a freeze-dried composition, and they may be sterilized or dissolved immediately before use in sterile distilled water for injection or some other solvent.

Otlier compositions for parenteral administration include liquids for external use, and ointment, endermic liniments, inhale, spray, suppositories for rectal administration and pessaries for vaginal administration which comprise Tempol and are administered by methods known in the art.

Spray compositions may comprise additional substances other than diluents: e.g. stabilizing agents (e.g. sodium sulfite hydride), isotonic buffers (e.g. sodium chloride, sodium citrate or citric acid). For preparation of such spray compositions, for example, the method described in U.S. Pat. No. 2,868,691 or No. 3,095,355 may be used. Briefly, a small aerosol particle size useful for effective distribution of the medicament may be obtained by employing self-propelling compositions containing the drugs in micronized form dispersed in a propellant composition. Effective dispersion of the finely divided drug particles may be accomplished with the use of very small quantities of a suspending agent, present as a coating on the micronized drug particles. Evaporation of the propellant from the aerosol particles after spraying from the aerosol container leaves finely divided drug particles coated with a fine film of the suspending agent. In the micronized form, the average particle size is less than about 5 microns. The propellant composition may employ, as the suspending agent, a fatty alcohol such as oleyl alcohol. The minimum quantity of suspending agent is approximately 0.1 to 0.2 percent by weight of the total composition. The amount of suspending agent is preferably less than about 4 percent by weight of the total composition to maintain an upper particle size limit of less than 10 microns, and preferably 5 microns. Propellants that may be employed include hydrofluoroalkane propellants and chlorofluorocarbon propellants. Dry powder inhalation may also be employed.

### EXAMPLE 1

A 70-kilogram patient exhibiting symptoms of dementia and for whom a diagnosis of Alzheimer's disease is suspected is administered a dose of 1500 mg of Tempol per day for 180 days. This may be administered in a single dose, or may be administered as a number of smaller doses over a 24-hour period: for example, three 500-mg doses at eight-hour intervals. Following treatment, the circulating serum protein levels of serum amyloid P-component precursor, serum amyloid A-1 protein precursor, JAK-1, cathepsin B, eukaryotic translation initiation factor 4E member 2, cyclooxygenase-1, and caspase 3 are reduced. Furthermore, the serum protein levels of eukaryotic translation initiation factor 4E binding protein are increased.

### EXAMPLE 2

A 70-kilogram patient with familial risk factors for Alzheimer's disease but exhibiting no symptoms of dementia is administered a dose of 1500 mg of Tempol per day for 180 days. This may be administered in a single dose, or may be administered as a number of smaller doses over a 24-hour period: for example, three 500-mg doses at eight-hour intervals. Following treatment, the circulating serum protein levels of serum amyloid P-component precursor, serum amyloid A-1 protein precursor, JAK-1, and cathepsin B, eukaryotic translation initiation factor 4E member 2, cyclooxygenase-1, and caspase 3 are reduced. Furthermore, the serum protein levels of eukaryotic translation initiation factor 4E binding protein are increased.

In addition to the protocols described above for the prophylaxis and treatment of Alzheimer's disease, the use of Tempol in the manner described above is also contemplated for the prophylaxis and treatment of other amyloid-related diseases, including but not limited to rheumatoid arthritis, familial Mediterranean fever, Parkinson's disease, end-stage renal disease, juvenile chronic arthritis, psioratic arthropathy, ankylosing spondylitis, Behcet's syndrome, reactive artlritis and Crohn's disease.

What is claimed is:

1. A method for reducing serum levels of one or more amyloid proteins, comprising:
identifying an individual in need of reducing serum amyloid protein levels; and
administering to that individual an effective amount of a nitroxide antioxidant.

2. The method of claim 1, wherein the nitroxide antioxidant is 4-hydroxy-2,2,6,6-tetramethylpiperidine-1-oxyl.

3. The method of claim 1, wherein the amyloid proteins are selected from a group consisting of serum amyloid P component precursor and serum amyloid A-1 protein precursor.

4. The method of claim 1, wherein the effective amount of a nitroxide antioxidant is within a range of 0.01-300 mg/kg.

5. The method of claim 1, wherein the effective amount of a nitroxide antioxidant is within a range of 0.1-250 mg/kg.

6. The method of claim 1, wherein the effective amount of a nitroxide antioxidant is within a range of 1-200 mg/kg.

7. The method of claim 1, wherein the effective amount of a nitroxide antioxidant is within a range of 2-150 mg/kg.

8. The method of claim 1, wherein the effective amount of a nitroxide antioxidant is within a range of 5-125 mg/kg.

9. The method of claim 1, wherein the effective amount of a nitroxide antioxidant is within a range of 7-100 mg/kg.

10. The method of claim 1, wherein the effective amount of a nitroxide antioxidant is within a range of 10-75 mg/kg.

11. The method of claim 1, wherein the effective amount of a nitroxide antioxidant is within a range of 15-30 mg/kg.

12. A method for inhibiting the progression of disease associated with an amyloid protein, comprising:
identifying an individual affected by or at risk for amyloid protein-associated disease; and
administering to that individual an amount of a nitroxide antioxidant effective to alter expression of a gene associated with the amyloid protein-related disease.

13. The method of claim 12, wherein the amyloid protein-associated disease is Alzheimer's disease.

14. The method of claim 12, wherein the amyloid protein-associated disease is selected from the group consisting of rheumatoid arthritis, familial Mediterranean fever, Parkinson's disease, end-stage renal disease, juvenile chronic arthritis, psioratic arthropathy, ankylosing spondylitis, Behcet's syndrome, reactive arthritis, and Crohn's disease.

15. The method of claim 12, wherein the nitroxide antioxidant is 4-hydroxy-2,2,6,6-tetramethylpiperidine-1-oxyl.

16. The method of claim 12, wherein the expression level of the gene is reduced.

17. The method of claim 16, wherein the gene is selected from a group consisting of genes encoding serum amyloid P-component precursor, serum amyloid A-1 protein precursor, JAI-1, cathepsin B, eukaryotic translation initiation factor 4E member 2, cyclooxygenase-1, and caspase 3.

18. The method of claim 12, wherein the expression level of the gene is increased.

19. The method of claim 18, wherein the gene is eukaryotic translation initiation factor 4E binding protein.

20. The method of claim 12, wherein the effective amount of a nitroxide antioxidant is within a range of 0.01-300 mg/kg.

21. The method of claim 12, wherein the effective amount of a nitroxide antioxidant is within a range of 0.1-250 mg/kg.

22. The method of claim 12, wherein the effective amount of a nitroxide antioxidant is within a range of 1-200 mg/kg.

23. The method of claim 12, wherein the effective amount of a nitroxide antioxidant is within a range of 2-150 mg/kg.

24. The method of claim 12, wherein the effective amount of a nitroxide antioxidant is within a range of 5-125 mg/kg.

25. The method of claim 12, wherein the effective amount of a nitroxide antioxidant is within a range of 7-100 mg/kg.

26. The method of claim 12, wherein the effective amount of a nitroxide antioxidant is within a range of 10-75 mg/kg.

27. The method of claim 12, wherein the effective amount of a nitroxide antioxidant is within a range of 15-30 mg/kg.

28. A method for treating Alzheimer's disease, comprising:
administering to an Alzheimer's patient an amount of a nitroxide antioxidant effective to inhibit Alzheimer's plaque formation.

29. The method of claim 28, wherein the nitroxide antioxidant is administered in an amount effective to reduce serum levels of at least one protein related to plaque formation.

30. The method of claim 29, wherein the protein related to plaque formation is selected from a group consisting of serum amyloid P-component precursor, serum amyloid A-1 protein precursor, JAK-1, cathepsin B, eukaryotic translation initiation factor 4E member 2, cyclooxygenase-1, and caspase 3.

31. The method of claim 28, wherein the nitroxide antioxidant is administered in an amount effective to increase serum levels of at least one protein related to plaque formation.

32. The method of claim 31, wherein the protein related to plaque formation is eukaryotic translation initiation factor 4E binding protein.

33. The method of claim 28, wherein the effective amount of a nitroxide antioxidant is within a range of 0.01-300 mg/kg.

34. The method of claim 28, wherein the effective amount of a nitroxide antioxidant is within a range of 0.1-250 mg/kg.

35. The method of claim 28, wherein the effective amount of a nitroxide antioxidant is within a range of 1-200 mg/kg.

36. The method of claim 28, wherein the effective amount of a nitroxide antioxidant is within a range of 2-150 mg/kg.

37. The method of claim 28, wherein the effective amount of a nitroxide antioxidant is within a range of 5-125 mg/kg.

38. The method of claim 28, wherein the effective amount of a nitroxide antioxidant is within a range of 7-100 mg/kg.

39. The method of claim 28, wherein the effective amount of a nitroxide antioxidant is within a range of 10-75 mg/kg.

40. The method of claim 28, wherein the effective amount of a nitroxide antioxidant is within a range of 15-30 mg/kg.

41. Use of a nitroxide antioxidant in the preparation of a medicament for reducing serum amyloid protein levels.

42. Use of a nitroxide antioxidant in the preparation of a medicament for altering intracellular levels of one or more proteins associated with an amyloid protein-associated disease.

43. Use of a nitroxide antioxidant in the preparation of a medicament for treating Alzheimer's disease.

44. Use of a nitroxide antioxidant in the preparation of a medicament for inhibiting Alzheimer's plaque formation.
